(19) United States
(12) Patent Application Publication
(10) Pub. No.: US 2017/0035724 A1
(43) Pub. Date: Feb. 9, 2017

(54) MAMMALIAN SIRT1 ACTIVATOR

(71) Applicant: Chang Gung University, Taoyuan City (TW)

(72) Inventors: Chin-Chuan CHEN, Taoyuan City (TW); Yann-Lii LEU, Taoyuan City (TW); Tsong-Long HWANG, Taoyuan City (TW); Jia-You FANG, Taoyuan City (TW); Tai-Long PAN, Taoyuan City (TW)

(21) Appl. No.: 14/885,645

(22) Filed: Oct. 16, 2015

(30) Foreign Application Priority Data
Aug. 7, 2015 (TW) 104125781

(51) Int. Cl. A61K 31/353; A61K 36/487

(52) U.S. Cl. CPC A61K 31/353; A61K 36/487

(57) ABSTRACT

A method for activating mammalian SIRT1 in a cell comprising:

providing a compound of formula (1):

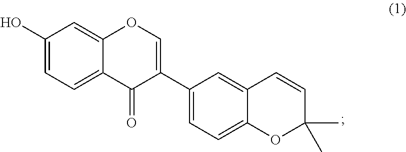 [SMILES: CC1(C)C=Cc2cc(-c3coc4cc(O)ccc4c3=O)ccc2O1]

and
incubating the cell with the compound. Also provided is a method for extending life span of a subject comprising: providing the same compound; and administering the compound to the subject. Yet also provided is a method for treating a mammalian SIRT1 inactivation-related disease in a subject comprising: providing the same compound; and administering the compound to the subject.

### CROSS REFERENCE

This non-provisional application claims priority from Taiwan Patent Application NO. 104125781, filed on 7 Aug. 2015, the content thereof is incorporated by reference herein.

### FIELD OF THE INVENTION

The present invention relates to an activator, and more particularly to a mammalian SIRT1 activator.

### BACKGROUND OF THE INVENTION

Mammalian SIRT1 is a member of the sirtuin family. SIRT1 is a nicotinamide adenine dinucleotide (NAD)-dependent deacetylase, and can remove the acetyl group of a histone protein or a non-histone protein. Accordingly, SIRT1 is involved in various physiological phenomena, such as regulation of gene expression, metabolism, and aging. Specifically, after the nicotinamide removal of SIRT1 from nicotinamide adenine dinucleotide, the acetyl group of the substrate is shifted to the nicotinamide-removed nicotinamide adenine dinucleotide. The substrate activated by SIRT1 continuously increases in number, and contains tumor suppressor protein p53, any member of FoxO family, HES1, HEY2, PPARγ, CTIP2, p300, PGC-1α, or NF-κB.

Up to now, there are few known SIRT1 activators, e.g. resveratrol, SRT1720, and SRT2379. Therefore, it is desirable to develop a novel SIRT1 activator for the application of related physiological phenomena.

### SUMMARY OF THE INVENTION

An objective of the present invention is to provide a method for activating mammalian SIRT1 in a cell. The activating method comprises:

providing a compound of formula (1):

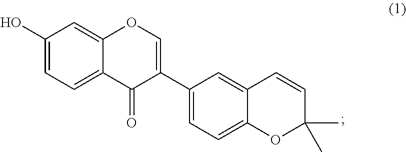 [SMILES: CC1(C)C=Cc2cc(-c3coc4cc(O)ccc4c3=O)ccc2O1]

and

incubating the cell with the compound.

Another objective of the present invention is to provide a method for extending life span of a subject. The activating method comprises:

providing a compound of formula (1):

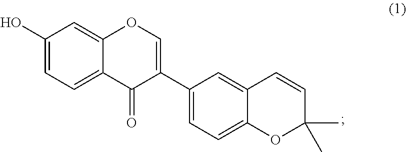 [SMILES: CC1(C)C=Cc2cc(-c3coc4cc(O)ccc4c3=O)ccc2O1]

and

administering the compound to the subject.

Another yet objective of the present invention is to provide a method for treating a mammalian SIRT1 inactivation-related disease in a subject. The treating method comprises:

providing a compound of formula (1):

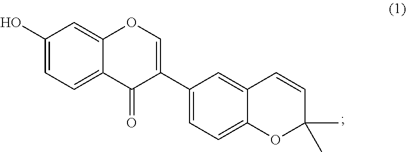 [SMILES: CC1(C)C=Cc2cc(-c3coc4cc(O)ccc4c3=O)ccc2O1]

and

administering the compound to the subject.

### BRIEF DESCRIPTION OF THE DRAWINGS

FIG. 1 is a chart illustrating the extraction of corylin from Psoralea corylifolia;

FIG. 2 is a graph illustrating the replicative life span of a corylin-treated yeast cell;

FIG. 3 is a graph illustrating the effect of various concentrations corylin on replicative life span of a yeast cell;

FIG. 4A shows polymerase chain reaction results illustrating the PNC1 transcription in a corylin-treated yeast after various treatment periods;

FIG. 4B shows real-time polymerase chain reaction results illustrating the PNC1 transcription in a corylin-treated yeast cell after various treatment periods;

FIG. 4C shows Western blotting results illustrating the PNC1 translation in a corylin-treated yeast cell after various treatment periods;

FIG. 5A shows Western blotting results illustrating the mammalian SIRT1 expression in a human osteosarcoma U2OS cell treated with corylin after various treatment periods;

FIG. 5B shows Western blotting results illustrating the p53 acetylation in a human lung cancer A549 cell treated with corylin in various concentrations;

FIG. 5C shows Western blotting results illustrating the p53 acetylation in a human osteosarcoma U2OS cell treated with corylin in various concentrations; and

FIG. 6 shows Western blotting results illustrating the p53 deacetylation in a human lung cancer A549 cell treated with corylin and nicotinamide.

### DETAILED DESCRIPTION OF THE INVENTION

The detailed description and preferred embodiments of the invention will be set forth in the following content, and provided for people skilled in the art so as to understand the characteristics of the invention.

In the present invention, a compound of formula (1) isolated from Psoralea corylifolia can lengthen yeast's lifespan. Based on this foundation, the present invention also discloses the compound can activate mammalian SIRT1. It is noted that the compound is represented by formula (1):

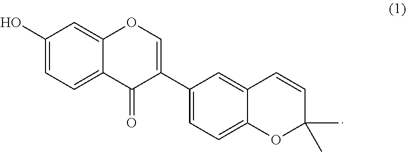 [SMILES: CC1(C)C=Cc2cc(-c3coc4cc(O)ccc4c3=O)ccc2O1]

This compound is scientifically named corylin. If not expressly indicated otherwise in the following description, the terms “a compound of formula (1)” and “corylin” used in the content are intended to convey the same meaning.

A first embodiment of the present invention discloses a method for activating mammalian SIRT1 in a cell, the method comprising: providing corylin; and incubating the cell with the corylin. In the cell, the corylin activates mammalian SIRT1 through the activation of an upstream of mammalian SIRT1. Furthermore, the corylin may be obtained via chemical synthesis or extraction from nature, such as extraction from Psoralea corylifolia.

As described in Cell Metab. 2013; 18(3): 416-30, activation of SIRT1 extends a mouse's lifespan. Since corylin can activate mammalian SIRT1, the compound may be used for lifespan lengthening. Therefore, a second embodiment of the present invention discloses a method for extending life span of a subject, the method comprising: providing corylin; and administering the corylin to the subject. Specifically, the corylin can be made in a medicine for the extension of the subject's life span. In the subject, the corylin activates mammalian SIRT1 so that the life span is extended. Moreover, the corylin may be obtained via chemical synthesis or extraction from nature, such as extraction from Psoralea corylifolia.

As described in Future Cardiol. 2012; 8(1): 89-100, resveratrol can treat a cardiovascular disease by activating mammalian SIRT1. As further described in Cell Metab. 2012; 16(2): 180-8, inactivation of mammalian SIRT1 in an adipose tissue results in obesity. As yet further described in Cell. 2010; 142(2): 320-32, SIRT1 activates ADAM 10-a secretase in a mouse to repress Alzheimer's disease. As yet further described in Nature. 2007; 450(7170): 712-6, activation of mammalian SIRT1 is a strategy for the treatment of diabetes mellitus. Since corylin can activate mammalian SIRT1, it can be used for the treatment of a mammalian SIRT1 inactivation-related disease.

Accordingly, a third embodiment of the present invention discloses a method for treating a mammalian SIRT1 inactivation-related disease in a subject, the method comprising: providing corylin; and administering the corylin to the subject. In the present embodiment, the disease may be cardiovascular disease, obesity, Alzheimer's disease, or diabetes mellitus. Specifically, the corylin can be made in a medicine for the disease treatment. In the subject, the corylin activates mammalian SIRT1 so as to treat the disease. Moreover, the corylin may be obtained via chemical synthesis or extraction from nature, such as extraction from Psoralea corylifolia.

The following examples are offered to further illustrate the present invention:

### EXAMPLE 1

### Extraction of Corylin

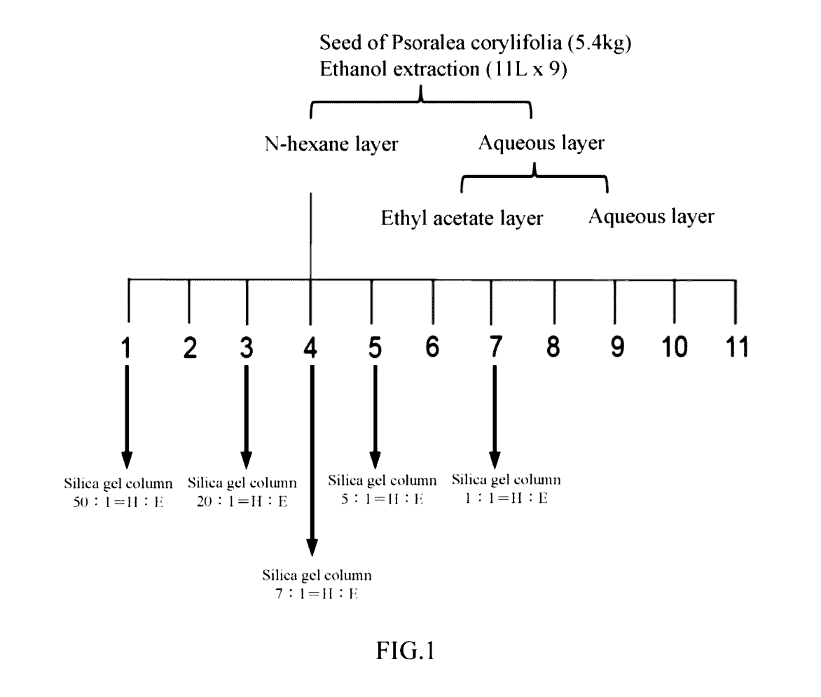

As shown in FIG. 1, Psoralea corylifolia seeds (5.4 kg) were pulverized, and then the pulverized seeds were extracted with ethanol. In the extraction process, the pulverized seeds were coldly soaked in 11 L ethanol for 4 times, and hotly soaked in 11 L ethanol at 70° C. for 4 hours for 5 times. After filtering the extract, the filtered extract was concentrated. The concentrated extract was partitioned with n-hexane and water to obtain an n-hexane layer and an aqueous layer. The aqueous layer was further partitioned with ethyl acetate to form an ethyl acetate layer and another aqueous layer; the n-hexane layer was further applied on a silica gel column, and then eluted with an n-hexane/ethyl acetate mixture in different concentration gradients to sequentially gain 11 fractions.

The first fraction was obtained with the elution of the n-hexane/ethyl acetate mixture in 50:1 and contained bakuchiol; the third fraction was obtained with the elution of the n-hexane/ethyl acetate mixture in 20:1 and contained isopsoralen; the forth fraction was obtained with the elution of the n-hexane/ethyl acetate mixture in 7:1 and contained psoralen; the fifth fraction was obtained with the elution of the n-hexane/ethyl acetate mixture in 5:1 and contained corylin; and the seventh fraction was obtained with the elution of the n-hexane/ethyl acetate mixture in 1:1 and contained psoralidin.

### EXAMPLE 2

### Effect of Corylin on Yeast's Replicative Life Span

A yeast colony was selected and cultured in a YEPD culture media. When the OD value of the media was at 0.6-0.8, 20 μl of the media was added to a YEPD solid media, and then 20-30 yeast cells were selected using a tetrad dissection microscope manipulation system. The selected yeast cells were cultured at 30° C. for 3-5 hours to divide into first generation yeast cells. Afterwards, the first generation yeast cells were cultured at 30° C. for 3-5 hours to divide into second generation yeast cells. The culturing step was repeated until final generation yeast cells which cannot divide anymore existed. Finally, the yeast cells' passage number was counted according to the total division number.

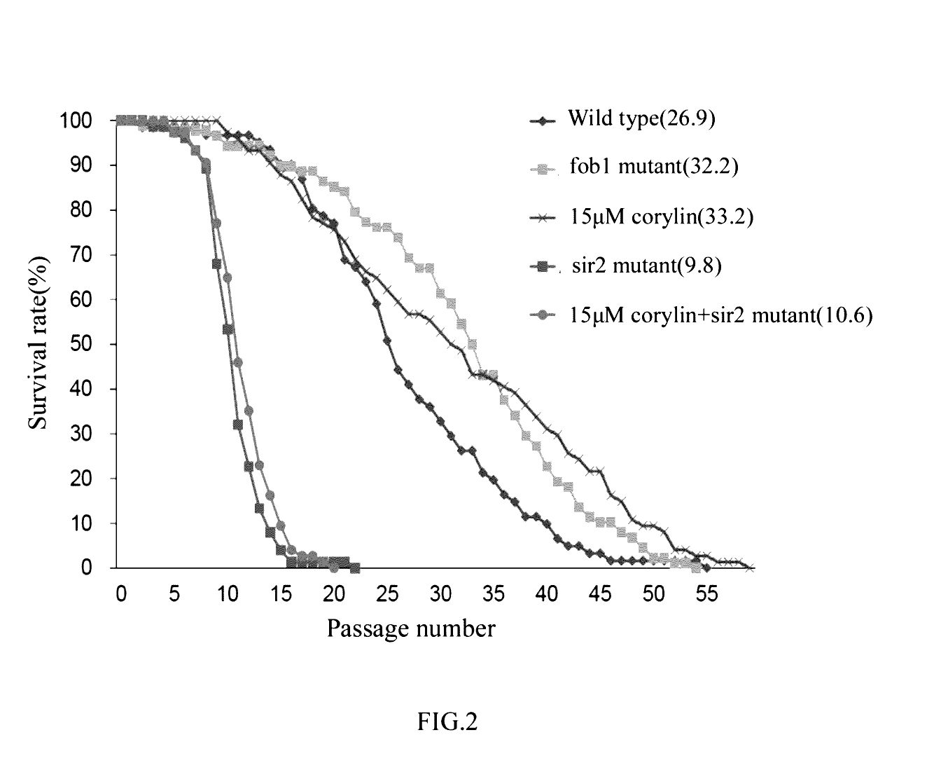

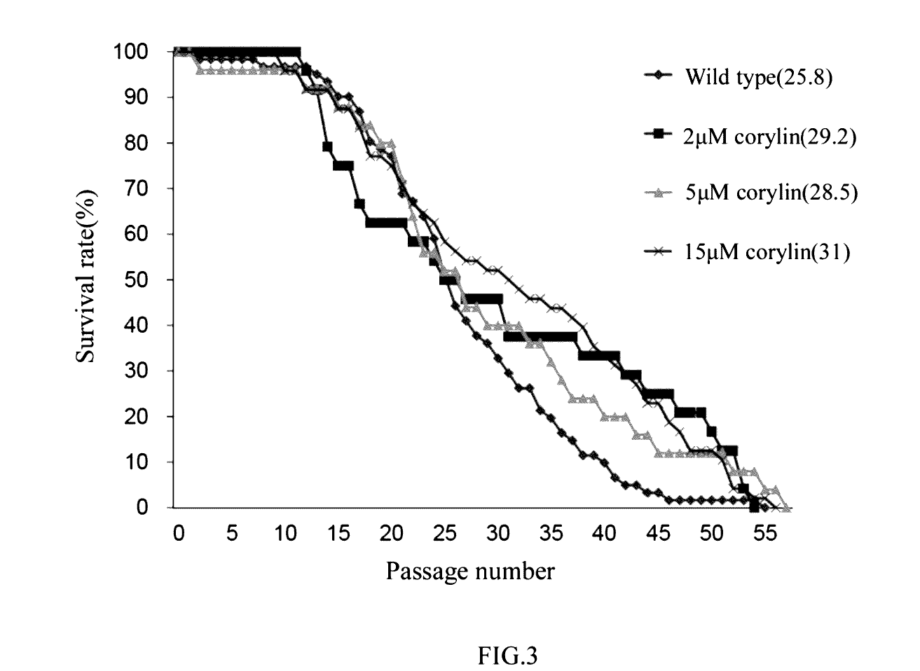

As shown in FIG. 2, the passage number of wild type yeast cells is less than that of wild type yeast cells treated with corylin (26.9 vs. 33.2); the passage number of sir2-mutant yeast cells is less than that of sir2-mutant yeast cells treated with corylin (9.8 vs. 10.6). This result implies that corylin can extend yeast cells' lifespan in a Sir2-dependent pathway. As also shown in FIG. 3, the effective concentration of corylin is 2-100 μM, and preferably is 2-15 μM.

### EXAMPLE 3

### Effect of Corylin on Activation of Yeast SIRr2

Yeast SIR2 is homology to mammalian SIRT1, and it is well known that Yeast SIR2 is associated with lifespan extension. See Genes Dev. 2000; 14(9): 1021-6.

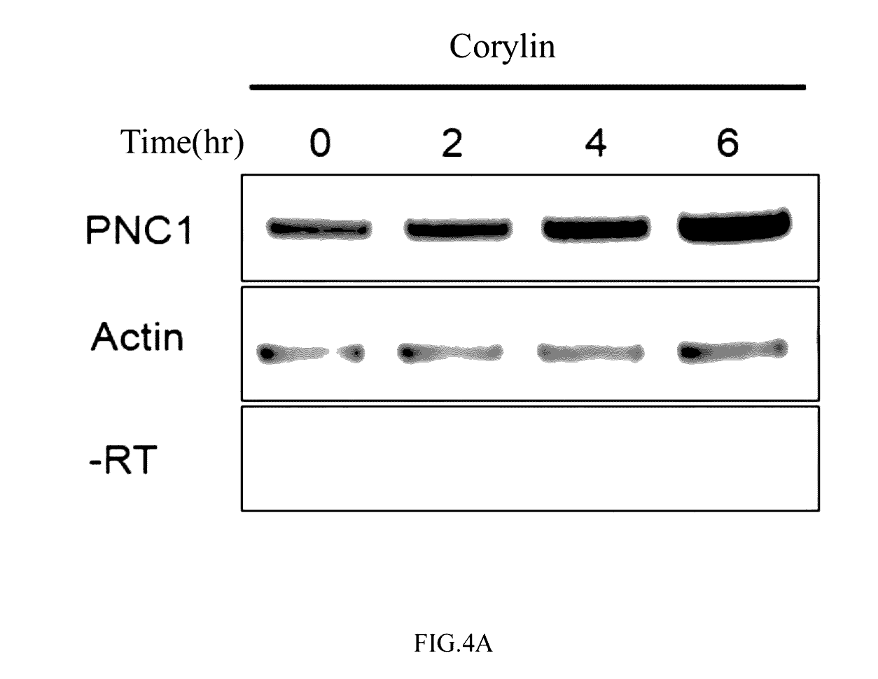

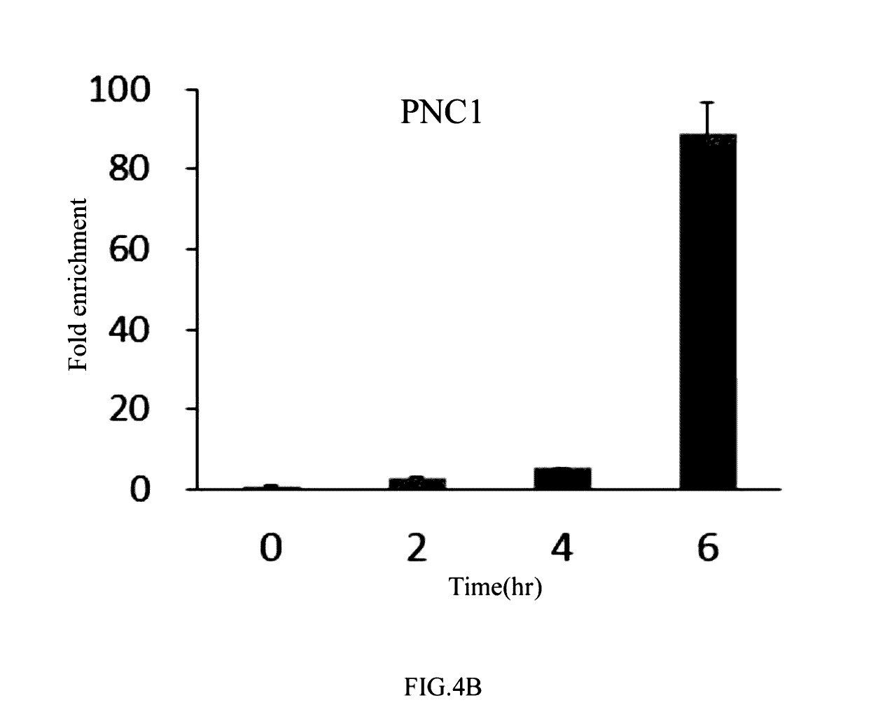

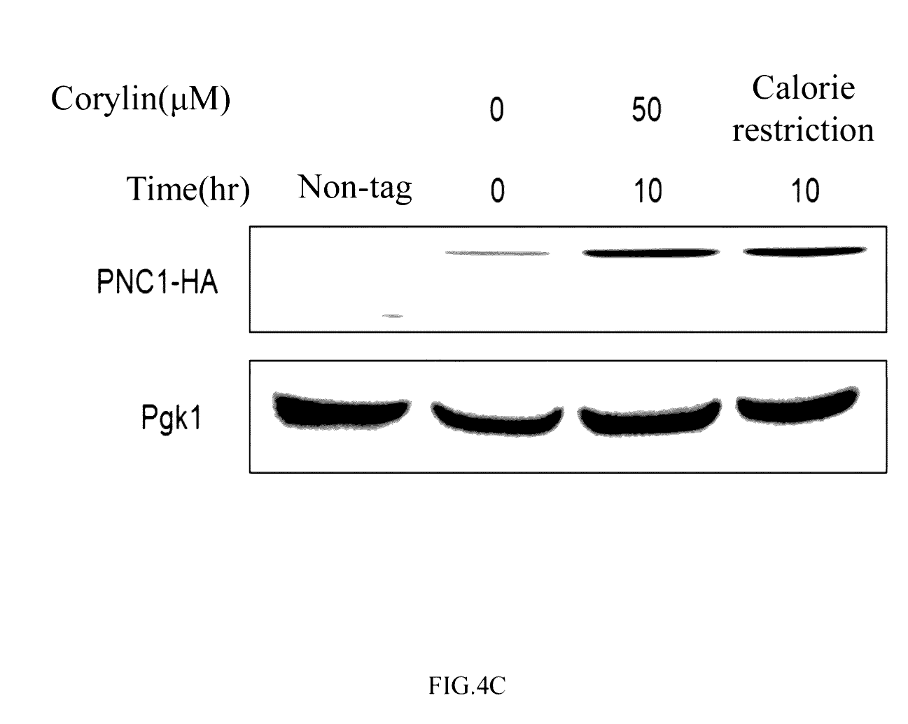

After yeast cells were treated with corylin for various periods, total RNA in the treated yeast cells was extracted. Then, the cDNA corresponding to the extracted RNA was formed using reverse transcription. Finally, the cDNA was analyzed using polymerase chain reaction and real-time polymerase chain reaction. As shown in FIGS. 4A and 4B, corylin can promote the transcription of PNC1, and the PNC1 transcription increases with the increasing of the treatment period.

To confirm the foregoing result, after yeast cells were treated with corylin in various concentrations for various periods, total protein therein was extracted and analyzed using Western blotting. As shown in FIG. 4C, as compared with the yeast cells with calorie restriction for 10 hours as positive control, the yeast cells treated with 50 μM corylin for 10 hours can obviously express Pnc1. This result indicates that corylin can promote the translation of PNC1.

Pnc1 is an upstream of yeast Sir2, and therefore corylin may activate yeast Sir2 through the activation of Pnc1.

### EXAMPLE 4

### Effect of Corylin on Activation of Mammalian SIRT1

Human osteosarcoma U2OS cells were seeded and cultured at 37° C. overnight, and then the cells were treated with corylin and cultured at 37° C. again. After various culturing periods, total protein in the treated cells was extracted and analyzed using Western blotting.

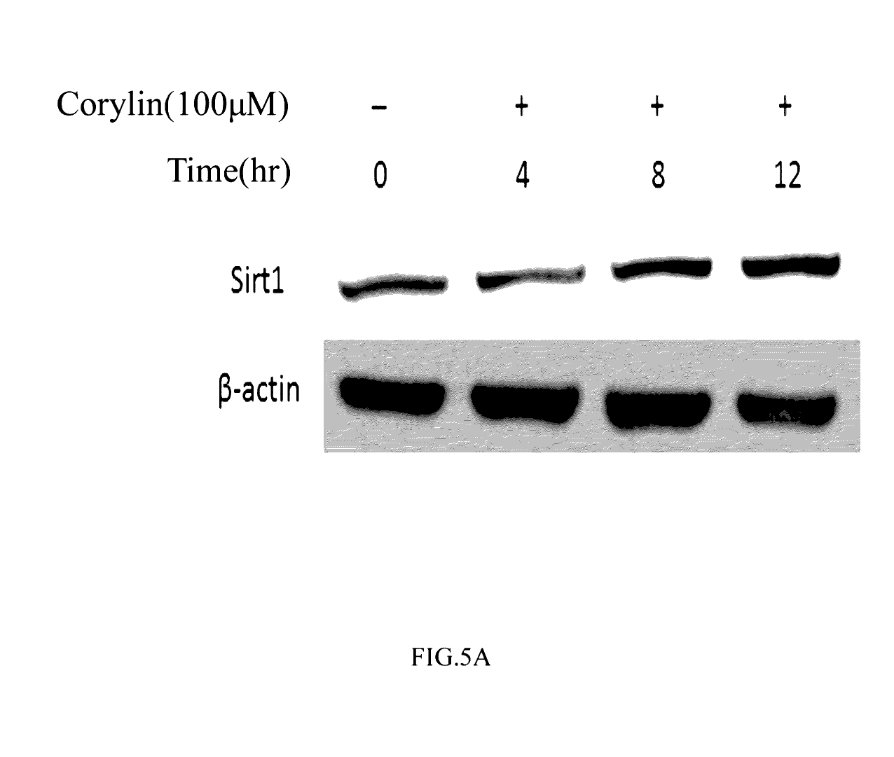

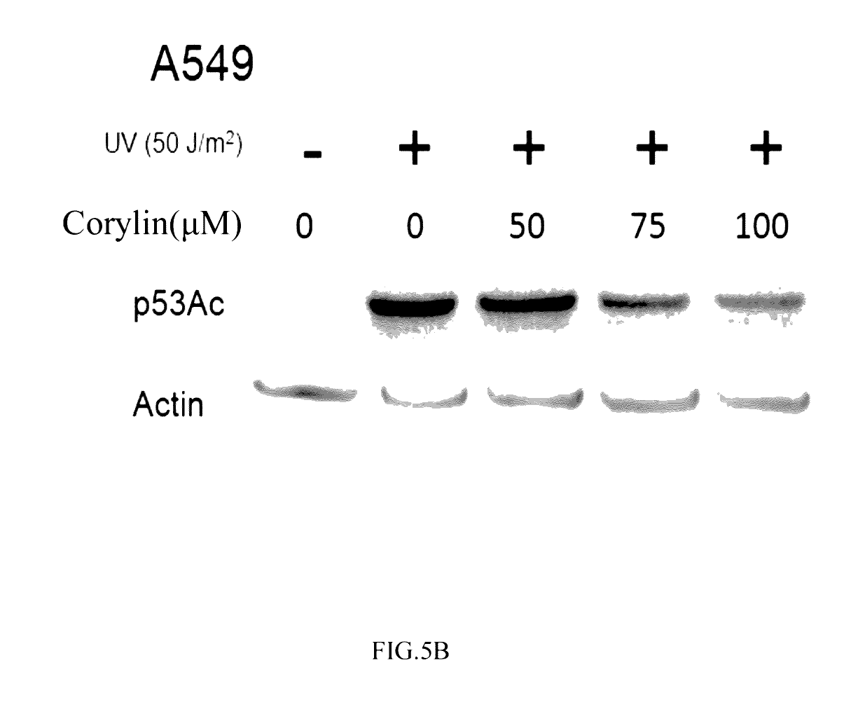

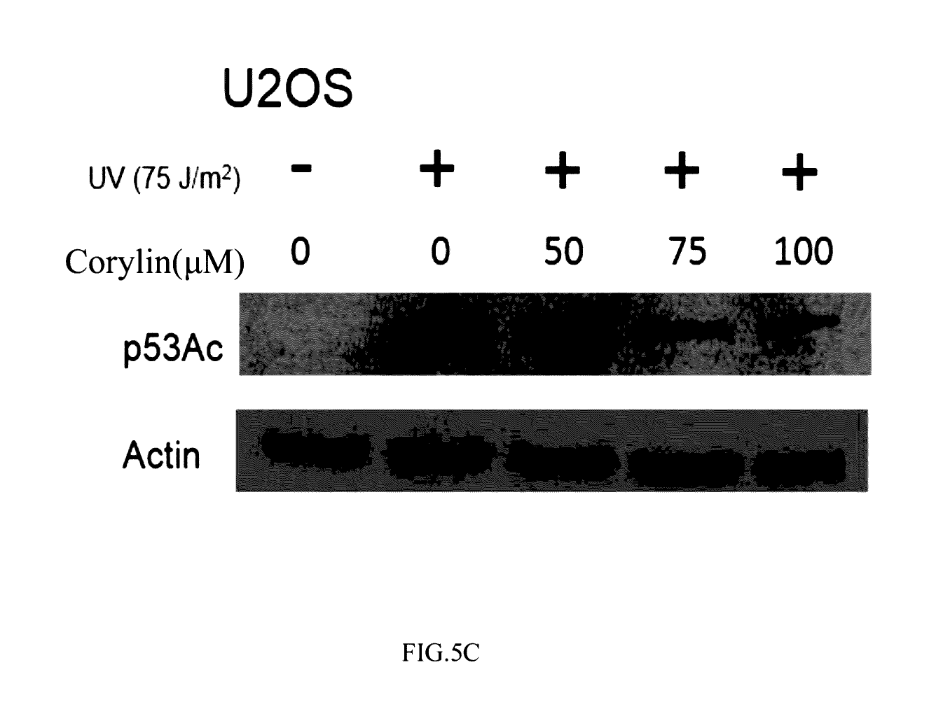

As shown in FIG. 5A, corylin can promote the expression of mammalian SIRT1, and the SIRT1 expression increases with the increasing of the culturing period.

After human lung cancer A549 cells or human osteosarcoma U2OS cells were cultured at 37° C. overnight, the cells were treated with various substances, and then cultured at 37° C. for 4 hours. After that, the cells were emitted with UV to acetylate p53, and cultured at 37° C. for 4 hours again. Finally, total protein extracted from the cells was analyzed using Western blotting.

As shown in FIGS. 5B and 5C, corylin can repress the acetylation of p53 induced by UV. According to the description in the “BACKGROUND OF THE INVENTION” section, p53 is a substrate for mammalian SIRT1. The result implies that corylin can promote the deacetylation of the downstream substrate for mammalian SIRT1. That is, corylin can activate the downstream substrate for mammalian SIRT1.

Nicotinamide is a mammalian SIRT1 inhibitor (Exp Hematol. 2012; 40(4): 342-55), and the compound is used to confirm the foregoing result. After human lung cancer A549 cells were cultured at 37° C. overnight, the cells were treated with a media containing nicotinamide, and then cultured at 37° C. for 1 hour. Corylin in various concentrations was added to the media, and the cells were cultured at 37° C. for 4 hours. After which, the cells were emitted with UV to acetylate p53, and cultured at 37° C. for 4 hours again. Finally, the total protein in the cells were extracted and analyzed using Western blotting.

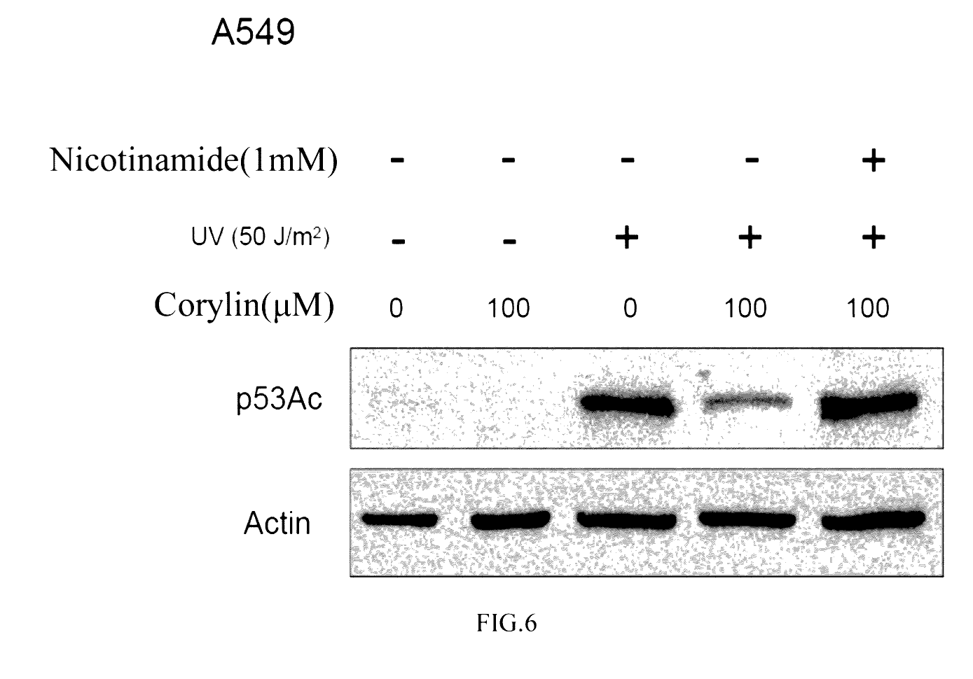

As shown in FIG. 6, nicotinamide can inhibit the p53 deacetylation caused by corylin. It is proven that mammalian SIRT1 is involved in the p53 deacetylation caused by corylin.

As shown above, corylin can activate mammalian SIRT1, and therefore the compound has potential as a mammalian SIRT1 activator.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

1. A method for activating mammalian SIRT1 in a cell, comprising:
providing a compound of formula (1): 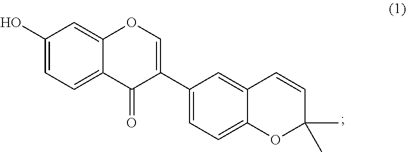 [SMILES: CC1(C)C=Cc2cc(-c3coc4cc(O)ccc4c3=O)ccc2O1]
and
incubating the cell with the compound.

2. The method as claimed in claim 1, wherein the compound is extracted from Psoralea corylifolia.

3. (canceled)

4. A method for extending life span of a subject, comprising:
providing a compound of formula (1): 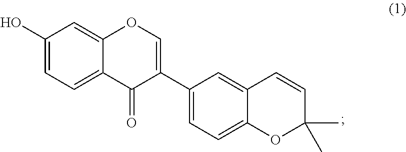 [SMILES: CC1(C)C=Cc2cc(-c3coc4cc(O)ccc4c3=O)ccc2O1]
and
administering the compound to the subject.

5. The method as claimed in claim 4, wherein the compound is extracted from Psoralea corylifolia.

6. The method as claimed in claim 4, wherein the compound activates mammalian SIRT1 in the subject.

7. A method for treating a mammalian SIRT1 inactivation-related disease in a subject, comprising:
providing a compound of formula (1): 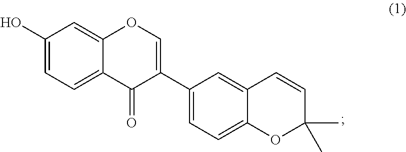 [SMILES: CC1(C)C=Cc2cc(-c3coc4cc(O)ccc4c3=O)ccc2O1]
and
administering the compound to the subject.

8. The method as claimed in claim 7, wherein the compound is extracted from Psoralea corylifolia.

9. The method as claimed in claim 7, wherein the disease is selected from the group comprising cardiovascular disease, obesity, Alzheimer's disease, and type 2 diabetes mellitus.
